(19) United States
(12) Patent Application Publication
(10) Pub. No.: US 2017/0000462 A1
(43) Pub. Date: Jan. 5, 2017

(54) ULTRASOUND IMAGING SYSTEM AND METHOD FOR ASSOCIATING A PHOTOGRAPHIC IMAGE WITH AN ULTRASOUND IMAGE

(71) Applicant: General Electric Company, Schenectady, NY (US)

(72) Inventors: Michael Joseph Washburn, Wauwatosa, WI (US); Nathan Robert Luttmann, Wauwatosa, WI (US); Dennis Jeffrey Meister, Livermore, CA (US)

(21) Appl. No.: 14/755,969

(22) Filed: Jun. 30, 2015

(51) Int. Cl. A61B 8/08; A61B 8/14; A61B 8/00; A61B 5/00

(52) U.S. Cl. CPC A61B 8/5215; A61B 5/0077; A61B 8/14; A61B 8/4416; A61B 8/463

(57) ABSTRACT

An ultrasound imaging system and method includes acquiring an ultrasound image, acquiring a photographic image, and associating, with a processor that is a component of the ultrasound imaging system, the photographic image with the ultrasound image. The ultrasound imaging system and method also includes display the ultrasound image and the photographic image on a display device after associating the photographic image with the ultrasound image.

### FIELD OF THE INVENTION

This disclosure relates generally to an ultrasound imaging system and method for acquiring an ultrasound image, acquiring a photographic image, and associating the photographic image with the ultrasound image.

### BACKGROUND OF THE INVENTION

The use of ultrasound imaging has recently become much more widespread. Ultrasound imaging is now commonly used to scan a wide variety of anatomies, and the ultrasound images are used to help diagnose a broad range of conditions. In many cases, it is difficult for the physician or radiologist to accurately understand and interpret ultrasound images unless additional contextual information is provided. For example, detailed information regarding the positioning of the probe at the time of acquisition may provide helpful contextual information to the physician or radiologist. Similarly, it may be helpful to view images of the exterior of the patient's body in the area of the scan in order to better understand how the ultrasound images relate to the external anatomy of the patient in one or more specific areas.

According to conventional techniques, the clinician may provide a written description of the patient's external anatomy. Written descriptions are inherently subjective, and different clinicians may use markedly different language when describing the same external anatomy. The use of written descriptions may therefore introduce unwanted variability into the interpretation of an ultrasound examination. Additionally, it takes additional time for the clinician to provide accurate written descriptions, which leads to longer scan times and less overall productivity.

In conventional techniques, the clinician may clarify an image with descriptive text describing the probe position. Or the clinician may overlay an anatomical graphic representing a portion of the patient's anatomy on the ultrasound image and then identify the current probe position with respect to the anatomical graphic. Both conventional techniques have significant shortcomings. They are manual processes, they are error prone, and they require additional time for the clinician to complete.

There is clearly a need for an improved technique of providing context to ultrasound images. For these and other reasons an improved ultrasound imaging system and method for associating a photographic image with an ultrasound image is desired.

### BRIEF DESCRIPTION OF THE INVENTION

The above-mentioned shortcomings, disadvantages, and problems are addressed herein, which will be understood by reading and understanding the following specification.

In an embodiment, a method of ultrasound imaging includes acquiring an ultrasound image with an ultrasound imaging system and acquiring a photographic image with a photographic imaging device. The method includes associating, with a processor that is a component of the ultrasound imaging system, the photographic image with the ultrasound image. The method includes displaying the ultrasound image and the photographic image on a display device after associating the photographic image with the ultrasound image.

In an embodiment, an ultrasound imaging system includes a probe, a display device, and a processor in electronic communication with the probe and the display device. The processor is configured to control the probe to acquire an ultrasound image, receive a photographic image from a photographic imaging device, and associate the photographic image with the ultrasound image. The processor is configured to display both the photographic image and the ultrasound image on the display device after associating the photographic image with the ultrasound image.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system and a photographic imaging device in accordance with an embodiment;

FIG. 2 is a schematic diagram of an ultrasound imaging system in accordance with an embodiment;

FIG. 3 is a flow chart of a method in accordance with an embodiment;

FIG. 4 is a timeline in accordance with an embodiment;

FIG. 5 is a schematic diagram of a DICOM header in accordance with an embodiment;

FIG. 6 is a schematic representation of a display in accordance with an embodiment; and

FIG. 7 is a schematic representation of a display in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical, and other changes may be made without departing from the scope of the embodiments. The following detailed description is, therefore, not to be taken as limiting the scope of the invention.

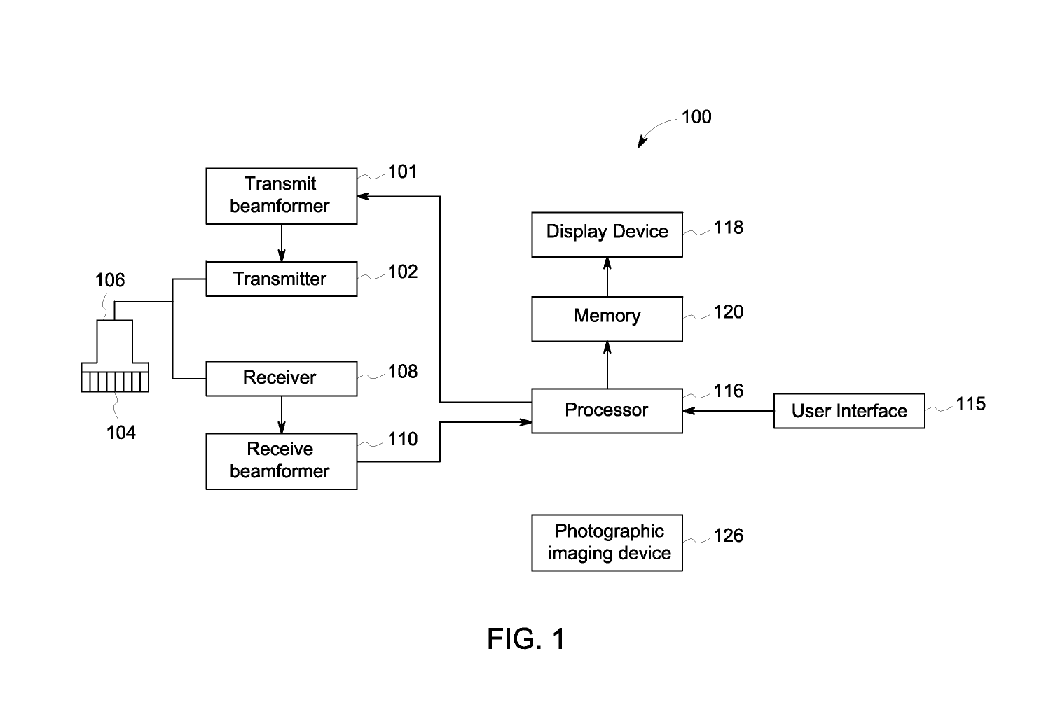

FIG. 1 is a schematic diagram of an ultrasound imaging system 100 and a photographic imaging device 126 in accordance with an embodiment. FIG. 1 shows the various components of the ultrasound imaging system 100. The ultrasound imaging system 100 includes a transmit beamformer 101 and a transmitter 102 that drive elements 104 within a probe 106 to emit pulsed ultrasonic signals into a body (not shown). The probe 106 may be any type of probe, including a linear probe, a curved array probe, a 1.25D array, a 1.5D array, a 1.75D array, or 2D array probe according to various embodiments. The probe 106 may be used to acquire 4D ultrasound data that shows information about how a volume changes over time. Each of the volumes may include a plurality of 2D images or slices. Still referring to FIG. 1, the pulsed ultrasonic signals are back-scattered from structures in the body, like blood cells or muscular tissue, to produce echoes that return to the elements 104. The echoes are converted into electrical signals, or ultrasound data, by the elements 104, and the electrical signals are received by a receiver 108. The electrical signals representing the received echoes are passed through a receive beamformer 110 that outputs ultrasound data. According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The terms “data” and “ultrasound data” may be used in this disclosure to refer to either one or more datasets acquired with an ultrasound imaging system. A user interface 115 may be used to control operation of the ultrasound imaging system 100. The user interface 115 may be used to control the input of patient data, or to select various modes, operations, parameters, and the like. The user interface 115 may include a one or more user input devices such as a keyboard, hard keys, a touch pad, a touch screen, a track ball, rotary controls, sliders, soft keys, or any other user input devices.

The ultrasound imaging system 100 also includes a processor 116 to control the transmit beamformer 101, the transmitter 102, the receiver 108, and the receive beamformer 110. The receive beamformer 110 may be either a conventional hardware beamformer or a software beamformer according to various embodiments. If the receive beamformer 110 is a software beamformer, it may comprise one or more of the following components: a graphics processing unit (GPU), a microprocessor, a central processing unit (CPU), a digital signal processor (DSP), or any other type of processor capable of performing logical operations. The receive beamformer 110 may be configured to perform conventional beamforming techniques as well as techniques such as retrospective transmit beamforming (RTB).

The processor 116 is in electronic communication with the probe 106. The processor 116 may control the probe 106 to acquire ultrasound data. The processor 116 controls which of the elements 104 are active and the shape of a beam emitted from the probe 106. The processor 116 is also in electronic communication with a display device 118, and the processor 116 may process the ultrasound data into ultrasound images for display on the display device 118. Also, for purposes of this disclosure, the phrase “acquiring an ultrasound image” may be used to describe the process of acquiring ultrasound data that will be used to generate an ultrasound image. For purposes of this disclosure, the term “electronic communication” may be used in this disclosure to include both wired and wireless connections. The processor 116 may include a central processing unit (CPU) according to an embodiment. According to other embodiments, the processor 116 may include other electronic components capable of carrying out processing functions, such as a digital signal processor, a field-programmable gate array (FPGA), a graphics processing unit (GPU), or any other type of processor. According to other embodiments, the processor 116 may include multiple electronic components capable of carrying out processing functions. For example, the processor 116 may include two or more electronic components selected from a list of electronic components including: a central processing unit (CPU), a digital signal processor (DSP), a field-programmable gate array (FPGA), and a graphics processing unit (GPU). According to another embodiment, the processor 116 may also include a complex demodulator (not shown) that demodulates the RF data and generates raw data. In another embodiment the demodulation may be carried out earlier in the processing chain. The processor 116 may be adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. Real-time frame or volume rates may vary based on the size of the region or volume from which data is acquired and the specific parameters used during the acquisition. The data may be stored temporarily in a buffer (not shown) during a scanning session and processed in less than real-time in a live or off-line operation. Some embodiments of the invention may include multiple processors (not shown) to handle the processing tasks. For example, a first processor may be utilized to demodulate and decimate the RF signal, while a second processor may be used to further process the data prior to display as an ultrasound image. For purposes of this disclosure, the term “acquiring an ultrasound image” may also be used to describe the process of acquiring ultrasound data and generating an ultrasound image based on the data. It should be appreciated that other embodiments may use a different arrangement of processors. For embodiments where the receive beamformer 110 is a software beamformer, the processing functions attributed to the processor 116 and the software beamformer hereinabove may be performed by a single processor such as the receive beamformer 110 or the processor 116. Or the processing functions attributed to the processor 116 and the software beamformer may be allocated in a different manner between any number of separate processing components.

According to an embodiment, the ultrasound imaging system 100 may continuously acquire ultrasound data, at a frame-rate of, for example, 10 Hz to 30 Hz. Images generated from the data may be refreshed at a similar frame-rate. Other embodiments may acquire and display data at different rates. For example, some embodiments may acquire ultrasound data at a frame rate of less than 10 Hz or greater than 30 Hz depending on the size of the volume and the intended application. A memory 120 is included for storing processed frames of acquired data. In an exemplary embodiment, the memory 120 is of sufficient capacity to store frames of ultrasound data acquired over a period of time at least several seconds in length. The ultrasound frames are stored in a manner to facilitate retrieval and display thereof according to the order or time of acquisition. The term “ultrasound frame” may be used to refer to either a frame of ultrasound data or a single image generate from a frame of ultrasound data. The memory 120 may comprise any known data storage medium.

Optionally, embodiments of the present invention may be implemented utilizing contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed by other or different mode-related modules by the processor 116 (e.g., B-mode, Color Doppler, M-mode, Color M-mode, spectral Doppler, Elastography, TVI, strain, strain rate, and the like) to form 2D or 3D images or data. For example, one or more modules may generate B-mode, color Doppler, M-mode, color M-mode, spectral Doppler, Elastography, TVI, strain, strain rate, combinations thereof, and the like. The ultrasound frames are stored and timing information indicating a time at which each ultrasound frame was acquired may be recorded in memory. The modules may include, for example, a scan conversion module to perform scan conversion operations to convert the ultrasound frames from beam space coordinates to display space coordinates. A video processor module may be provided that reads the image frames from a memory and displays the image frames in real-time while a procedure is being carried out on a patient. A video processor module may store the image frames in an image memory, from which the images are read and displayed.

A photographic imaging device 126 that is separate from the ultrasound imaging system is shown in FIG. 1. The photographic imaging device may be a camera or a camera-enabled device, such as a smartphone, a tablet, or any other device with a built-in camera or video recorder. The camera or video recorder may, for example, use a Charge-Coupled Device (CCD) or a Complementary Metal-Oxide-Semiconducter (CMOS) to capture one or more image frames. Cameras or video recorders using other types of image-capturing technology may be used according to other embodiments. The photographic imaging device 126 may be configured to acquire still pictures or video clips according to various embodiments. The video clip comprises a plurality of image frames that may be displayed in sequence in a manner similar to a movie. The photographic imaging device 126 may be adapted to transmit photographic images to the processor 116 via a cable, or the photographic imaging device 126 may be configured to transmit photographic images to the processor 116 via a wireless protocol, such as Bluetooth, WiFi, cellular, or any other wireless protocol.

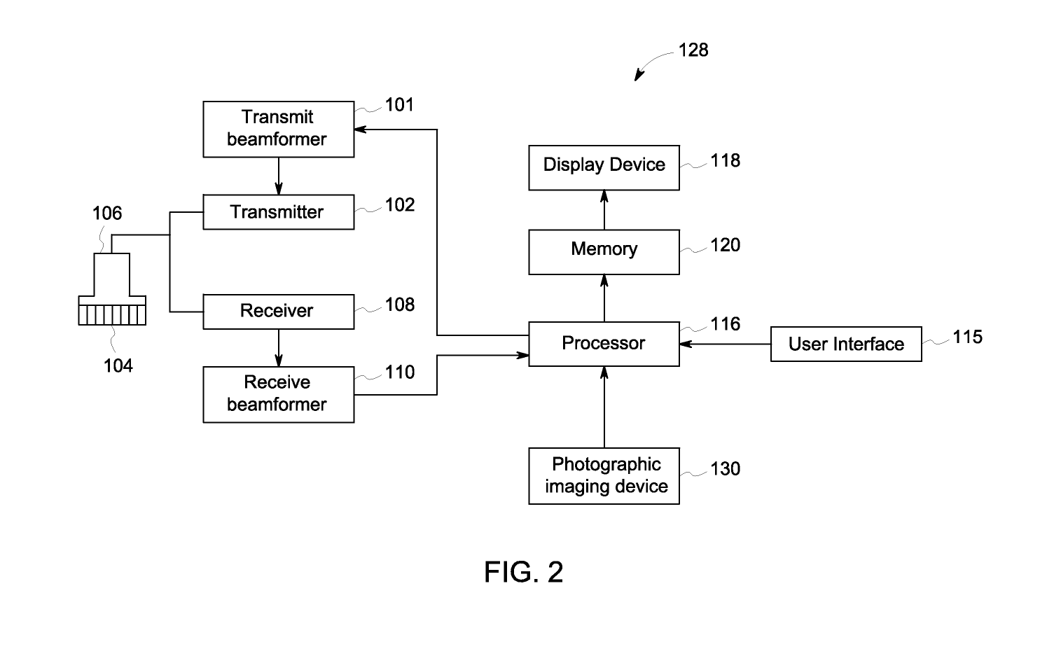

FIG. 2 is a schematic representation of an ultrasound imaging system 128 in accordance with an exemplary embodiment. Many of the elements represented in FIG. 2 are identical to elements that were previously described with respect to FIG. 1. Common reference numbers are used to identify identical elements in both FIGS. 1 and 2.

FIG. 2 includes a photographic imaging device 130. The photographic imaging device 130 may comprise a camera that is an integral component of the ultrasound imaging system 128. For example, the photographic imaging device 130 may be built into the ultrasound imaging system 100, the photographic imaging device 130 may be mounted on the ultrasound imaging system 100, or the photographic imaging device 130 may be mounted on a system arm attached to the ultrasound imaging system 100. The photographic imaging device 130 may also be attached to the probe 106 or built into the probe 106 according to other embodiments. It should be appreciated that the photographic imaging device 130 may be integrated into the ultrasound imaging system 100 according to other techniques in other embodiments. The photographic imaging device 130 may also be mounted to a cart configured to transport the ultrasound imaging system 100 in other embodiments. The photographic imaging device 130 may be configured to both acquire image frames and video clips according to an embodiment. The video clip may comprise a plurality of image frames, and the still picture may comprise a single image frame. The photographic imaging device 130 may comprise any type of camera or video recorder, and the photographic imaging device 130 may capture the images using CCD or CMOS technology, or the photographic imaging device 130 may use any other type of image-capturing technology.

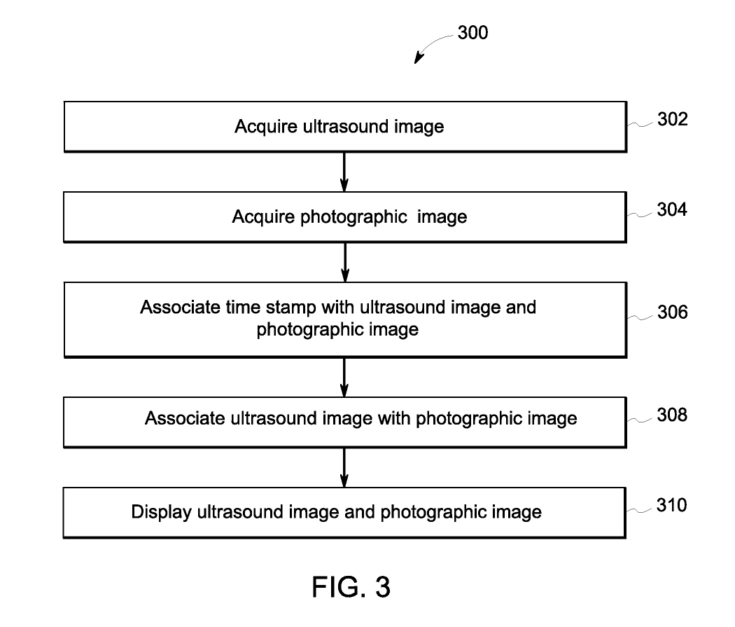

FIG. 3 is a flow chart of a method 300 in accordance with an exemplary embodiment. The individual blocks of the flow chart represent steps that may be performed in accordance with the method 300. Additional embodiments may perform the steps shown in a different sequence, and/or additional embodiments may include additional steps not shown in FIG. 3. The technical effect of the method 300 is associating the photographic image with the ultrasound image and displaying both the photographic image and the ultrasound image on a display device after associating the photographic image with the ultrasound image. The method 300 will be described according to an exemplary embodiment using the ultrasound imaging system 100.

At step 302, the processor 116 controls the probe 106 to acquire an ultrasound image. The ultrasound image may be either a 2D image or a 3D image. The ultrasound image may be a still image or an ultrasound clip. For purposes of this disclosure, the term “still image” may be used to refer to a single ultrasound frame, while the term “ultrasound clip” may be used to refer to a plurality of ultrasound frames acquired in sequence, each at a different point in time. When displayed, each of the ultrasound frames in an ultrasound clip is displayed in sequence, which allows the ultrasound clip to display motion in a manner similar to a movie. The ultrasound clip, which is also commonly referred to as a cine loop by those skilled in the art, may include either 2D or 3D ultrasound frames acquired over a period of time.

According to an embodiment, the processor 116 may associate a time stamp, indicating the time of acquisition, with each ultrasound frame acquired by the imaging system 100. For embodiments involving the acquisition of an ultrasound clip, the processor 116 may associate a time stamp with one or both of a starting time of the ultrasound clip and an ending time of the ultrasound clip, or the processor 116 may associate a time stamp with each of the individual ultrasound frames in the ultrasound clip.

At step 304, a photographic image is acquired with the photographic imaging device 126. According to an exemplary embodiment, the photographic imaging device 126 may be a stand-alone device such as a camera, a smartphone, or a tablet. Other embodiments may use a photographic imaging device, such as the photographic imaging device 130 shown in FIG. 2, that is integral with the ultrasound imaging system.

The photographic image may include a still picture or a video clip. For purposes of this disclosure, a still picture may include a single image frame, while a video clip may include a plurality of image frames that were acquired in sequence, each at a different point in time. When played, each of the image frames of the video clip is displayed in sequence, which allows the video clip to display motion in a manner similar to a movie.

At step 306, the processor 116 associates a time stamp with each image frame of the photographic image, and the processor 116 associates a time stamp with each ultrasound frame of the ultrasound image. According to an embodiment where the photographic image comprises a still picture, a single time stamp is associated with the still picture. The time stamp represents the time of acquisition of the still picture. According to an embodiment where the photographic image is a video clip, a time stamp, indicating the time of acquisition, may be associated with each image frame of the video clip. Likewise, if the ultrasound image comprises a single image frame, a single time stamp may be associated with the ultrasound image indicating the time of acquisition of the ultrasound image. And, correspondingly, if the ultrasound image comprises an ultrasound clip, a time stamp, indicating the time of acquisition, may be associated with each ultrasound frame of the ultrasound clip.

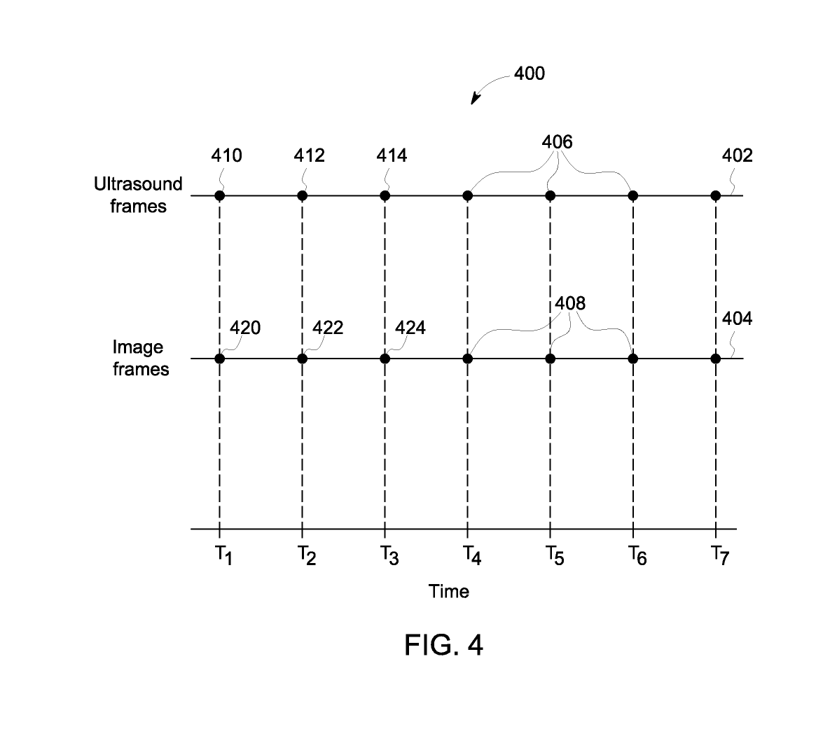

FIG. 4 is a schematic representation of a first timeline 402 and a second timeline 404. The first timeline 402 is a schematic representation of the acquisition of an ultrasound clip in according with an exemplary embodiment. The second timeline 404 is a schematic representation of the acquisition of a video clip in accordance with an exemplary embodiment.

The first timeline 402 includes a plurality of ultrasound frames 406 generated based on ultrasound data. The position of each ultrasound frame 406 on the first timeline 402 represents the time of acquisition for the particular ultrasound frame 406. Likewise, the second timeline 404 includes a plurality of image frames 408 acquired with the photographic imaging device 126. The position of each image frame 408 on the second timeline 404 represents the time of acquisition for each of the plurality of image frames 408. As mentioned previously, collectively, the plurality of ultrasound frames 406 forms an ultrasound clip, which, when viewed as a sequence, may be used to show motion in a manner similar to a movie. The plurality of image frames 406 collectively forms a video clip that may also be used to show motion when viewed in sequence.

During step 306, the processor 116 may be configured to associate a time stamp indicating a time of acquisition with each of the plurality of ultrasound frames 406. For example, a time stamp indicating a time T₁ may be associated with the ultrasound frame 410, a time stamp indicating the time T₂ may be associated with the ultrasound frame 412, a time stamp indicating the time T₃ may be associated with the ultrasound frame 414, and so forth.

Likewise, a time stamp, indicating a time of acquisition, may be associated with each of the plurality of image frames 408. For example, a time stamp indicating a time T₁ may be associated with the image frame 420, a time stamp indicating a time T₂ may be associated with the image frame 422, a time stamp indicating a time T₃ may be associated with the image frames 424, and so forth.

According to the exemplary embodiment described above, there is an ultrasound frame 406 acquired at the same time as each of the image frames 408. The time stamp associated with each ultrasound frame 406 and with each image frame 408 may be used to determine the particular ultrasound image frame 406 that corresponds to a specific image frame 408. For example, ultrasound frame 410 corresponds with image frame 420 because both were acquired at time T₁. In other embodiments, the time stamps for the image frames 408 may not be exactly the same as the time stamps for the ultrasound frame 406s. For embodiments where the time stamps are not exactly the same, the processor 116 may associate an image frame with an ultrasound frame by identifying the ultrasound frame with a time stamp that is closest to the time stamp of each particular photographic image.

At step 308, the processor 116 associates the ultrasound image with the photographic image. According to an embodiment, a clinician may first select a patient examination event with the user interface 115. The patient examination event may comprise an examination or a study. For example, if a workflow for a specific examination is selected, the workflow may guide the user to acquire all the required images for that particular patient examination event. The processor may automatically store both the ultrasound image and the photographic image with the same patient examination event. According to other embodiments, the user may manually determination to associate the photographic image with the ultrasound image after the acquisition of both images. For example, the clinician may manually select to associate both the ultrasound image and the photographic image with the same patient examination event after acquiring the ultrasound image and the photographic image.

For many embodiments, it may be desirable to transmit a report of the patient examination event from the ultrasound imaging system 100. For example, the report may be transmitted to a Picture Archiving and Communication System (PACS), or the report may be sent to a remote workstation for review and/or analysis. A communication standard, such as the Digital Imaging and Communications in Medicine, hereinafter DICOM, standard may be used when transmitting the report.

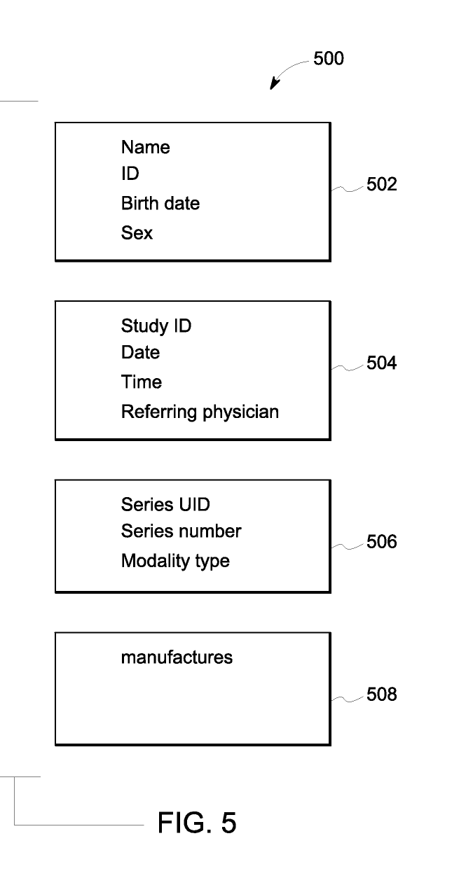

FIG. 5 is a schematic representation of a DICOM header 500 that may be associated with an image. DICOM is a standard for storing, handling, and transmitting medical images. In order to comply with the DICOM standard, a DICOM header, such as the DICOM header 500, is attached to the image. It should be appreciated that the DICOM header 500 shows only a limited subset of the DICOM elements that may be included in the DICOM header. The DICOM header 500 includes the following DICOM elements: a patient element 502, a study element 504, a series element 506, and an equipment element 508. The patient element 502 may include data identifying the patient such as name, ID, birth date, and sex. The study element 504 may include data such as the study ID, the date, the time of the study, and the referring physician. The series element 506 may include data such as a series UID, a series number, and a modality type. The equipment element 508 may include data identifying the medical diagnostic equipment used to acquire the image. Those skilled in the art will appreciate that the DICOM header may include DICOM elements other than or in addition to those described hereinabove.

The processor 116 may associate the ultrasound image with the photographic image by using DICOM header information, such as that shown in DICOM header 500. The processor 116 may also use information from a communication standard other than DICOM according to other embodiments. According to an embodiment, both the photographic image and the ultrasound image may be part of the same patient examination event, as indicated by the study element 504. One or more images, such as an ultrasound image and/or a photographic image, may be stored with a DICOM header as a DICOM object. For example, a first DICOM header may be associated with the photographic image and a second DICOM header may be associated with the ultrasound image. According to an embodiment, the first DICOM header may be permanently associated with the photographic image and the second DICOM header may be permanently associated with the ultrasound image. The DICOM header information in the DICOM header is used to link the ultrasound image and the photographic image to the patient examination event. Therefore, according to this exemplary embodiment, the photographic image will be permanently associated with the ultrasound image through DICOM header information. It is not possible for a typical clinician or physician to disassociate the DICOM header from the image. As such, associating the photographic image with the ultrasound image is very robust; it is preserved even if the examination is sent to multiple different workstations and/or storage locations. This provides a significant advantage compared to conventional techniques where the association between photographic image and ultrasound image can easily be lost.

According to other embodiments, the photographic image may be associated with the ultrasound image in ways other than with DICOM header information, or the photographic image may be associated with the ultrasound image in ways in addition to DICOM header information.

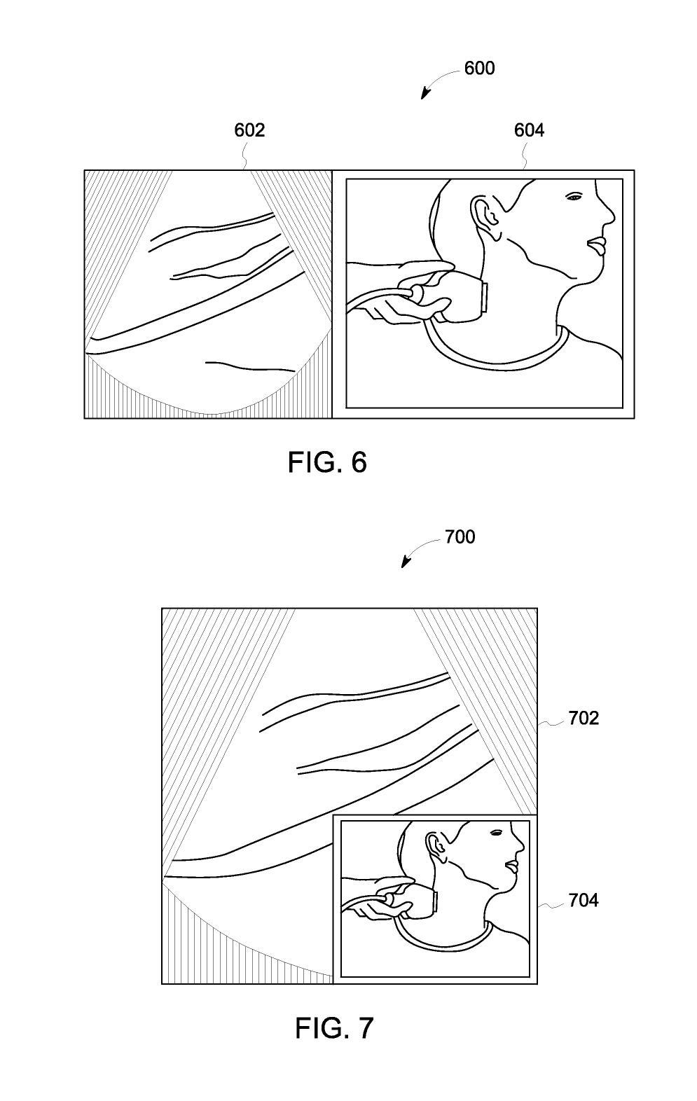

At step 310, the processor 116 controls the display of the ultrasound image and the photographic image on the display device 118. The processor 116 may display the ultrasound image and the photographic image in sequence. For example, the processor 116 may first display the photographic image to provide contextual information to the clinician or physician viewing the examination, and then the processor 116 may display the ultrasound image. In another embodiment, the processor 116 may display the photographic image at the same time as the ultrasound image on the display device. For example, the photographic image and the ultrasound image may be displayed side by side as shown in FIG. 6.

FIG. 6 is a schematic representation of a screenshot 600 in accordance with an exemplary embodiment. The screenshot may be displayed on a display device, such as the display device 118. The screenshot 600 includes an ultrasound image 602 and a photographic image 604. While FIG. 6 shows the ultrasound image 602 and the photographic image 604 displayed side by side, it should be appreciated that other embodiments may display the photographic image and the ultrasound image in different orientations. For example, the photographic image may be displayed either above or below the ultrasound image. Additionally, in other embodiments, the user may toggle between the ultrasound image and the photographic image in response to a user input on the user interface 115.

FIG. 6 will be discussed with respect to an exemplary embodiment where the ultrasound image 602 represents one ultrasound frame of an ultrasound clip and the photographic image 604 represents one image frame from a video clip. It should be appreciated that, when actually viewing the ultrasound clip and the video clip, the viewer will view all the image frames and all of the ultrasound frames, respectively, in series. More specifically, according to this exemplary embodiment, the video clip shows the position of the probe 106 with respect to the patient during the time period that the ultrasound clip was acquired.

The processor 116 displays or plays the video clip in time synchronization with the ultrasound clip by using the time stamps associated with the image frames and the ultrasound frames during step 306. This provides several benefits to the user. First, the user is able to see the exact position of the probe 106 with respect to the patient at each frame of the ultrasound clip. This enables the user to easily determine the orientation of the probe 106 with respect to the patient, which may affect the resulting ultrasound image. The information provided by the video clip allows the user to quickly determine if the probe 106 is positioned in a conventional/standardized orientation for a given protocol, or if the ultrasound image was acquired with the probe 106 in a non-standard position or orientation. Second, the video clip provides valuable position and orientation data to the user for acquisitions where the clinician needs to move the probe 106, such as when using a 2D probe to acquire a 3D dataset. For example, the clinician may be required to tilt, translate, or rotate the probe to acquire the 3D dataset. Displaying a time-synchronized video clip at the same time as the ultrasound clip allows the user to very easily determine the exact anatomy included in the 3D dataset and the exact position/orientation of the probe 106 at any particular frame of the ultrasound clip. This provides contextual information regarding the position of the probe 106 with respect to the patient at any particular time during the acquisition of the ultrasound clip. For example, the user may also freeze both the ultrasound clip and the video clip while maintaining the time synchronization. In other words, both the ultrasound clip and the video clip may both be stopped in response to the user initiating a freeze command. This allows the user a longer period of time to investigate a particular frame of the ultrasound clip. However, by stopping the video clip at the same time, the user still has access the contextual information provided by the video clip for the particular ultrasound frame under investigation.

While the above example described an embodiment where the ultrasound clip was acquired by moving the probe 106, it should be appreciated that the probe 106 may be kept substantially still while acquiring the ultrasound clip. This may be used, for instance, when acquiring an ultrasound clip to show moving anatomy, such as a heart.

FIG. 7 is a schematic representation of a screenshot 700 in accordance with an exemplary embodiment. The screenshot 700 includes an ultrasound image 702 and a photographic image 704. The photographic image 704 is positioned within the ultrasound image. For this reason, the photographic image 704 may be considered a sub-image of the ultrasound image 702. The user may be able to control the position of the photographic image 704 within the ultrasound image 702, such as by translating the position of the photographic image 704. The user may also toggle between a view showing the ultrasound image 702 and the photographic image 704 and a view showing the ultrasound image 702 without the photographic image 704 (not shown).

In other embodiments, the ultrasound image may be a still image and the photographic image may be a still picture. In embodiments where the ultrasound image is a still image, the photographic image may show the position of the probe 106 with respect to the patient's anatomy at the time when the ultrasound image was acquired.

In other embodiments, the photographic image 604, which may be a still picture or a video clip, may comprise an external view of the patient's anatomy at a location corresponding to the ultrasound image 602. For example, the photographic image 604 may show the exterior of the patient at or near the location where the probe 106 was placed to acquire the ultrasound image 602. This provides the user with strong contextual information that may be used to interpret the ultrasound image and/or help provide a diagnosis. For example, the photographic image may provide contextual information regarding the size, shape, and position of any visible symptoms such as: information regarding localized swelling; and information regarding bruising, information regarding any cuts, scraps, scars, or other types of trauma to the patient's exterior. Additionally, the video clip may allow the patient to demonstrate any limitations or other symptoms that could potentially help with the diagnosis. As discussed previously, the photographic image 604 is associated with the ultrasound image 602 by the processor 116 located within the ultrasound imaging system 100. This provides a technique to permanently associate the photographic image 604 with the ultrasound image 602, which enables all future viewers of the ultrasound image 602, even those on remote workstations, to benefit from the contextual information provided by the photographic image 604.

According to an embodiment, the user may edit the photographic image 604 using any of the editing functions available on the ultrasound imaging system 100 that would typically be used for editing the ultrasound image 602. For example, the user may adjust a zoom level, crop the image, adjust a level of contrast, rotate the image, adjust the window width or window level, or perform any other image editing function. According to some embodiments, the user may be able to edit these image attributes through the user interface 115 of the ultrasound imaging system 100 or while viewing the ultrasound image 602 and the photographic image 604 on a remote workstation.

In other embodiments, the photographic imaging device 126 may be used to capture a photographic image of an optical code, such as a bar code, a QR code, or any other type of optically encoded information. The processor 116 may associate the optical code with the ultrasound image, or the processor 116 may use the information contained in the optical code to populate one or more data fields. For example, the optical code may include patient-identifying information, such as name, gender, height, weight, etc., or information about the equipment used during the scan. Additionally, the optical code may include information specifying previous procedures or known medical issues associated with the patient. The processor 116 may, for instance, read the information in the optical code and use the information to populate data fields that are then associated with one or more ultrasound images acquired during a patient examination event.

According to other embodiments, the photographic imaging device 126 may be used to capture a photographic image in order to provide context for the examination. For example, the photographic image may include a picture or a video of a patient report, a picture or video of lab results from the patient, or a picture or video of equipment used as part of the examination. The photographic image may include a picture or a video showing the settings of equipment used to acquire the image, or it may include a picture or video of the output of other medical equipment used to collect medical information associated with the patient. It should be appreciated that the photographic image may include or represent other types of information or subject matter according to other embodiments.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of ultrasound imaging comprising:
acquiring an ultrasound image with an ultrasound imaging system;
acquiring a photographic image with a photographic imaging device;
associating, with a processor that is a component of the ultrasound imaging system, the photographic image with the ultrasound image; and
displaying the ultrasound image and the photographic image on a display device after associating the photographic image with the ultrasound image.

2. The method of claim 1, wherein associating the photographic image with the ultrasound image comprises establishing a patient examination event, and linking both the photographic image and the ultrasound image to the patient examination event.

3. The method of claim 2, further comprising using DICOM header information to link both the photographic image and the ultrasound image to the patient examination event.

4. The method of claim 3, wherein both the photographic image and the ultrasound image are stored as a single DICOM object.

5. The method of claim 3, wherein the photographic image is stored as a first DICOM object with a first DICOM header, and the ultrasound image is stored as a second DICOM object with a second DICOM header.

6. The method of claim 1, wherein the photographic image comprises a still picture.

7. The method of claim 6, wherein the still picture comprises an external view of a patient's anatomy at a location corresponding to the ultrasound image, and
wherein the still picture provides additional contextual information when viewed with the ultrasound image.

8. The method of claim 6, wherein the still picture comprises an image of a probe with respect to a patient's anatomy that was used while acquiring the ultrasound image.

9. The method of claim 1, wherein the photographic image comprises a video clip.

10. The method of claim 9, wherein the ultrasound image comprises an ultrasound clip, and wherein the video clip comprises a video showing the probe with respect to a patient's anatomy during the process of acquiring the ultrasound clip.

11. The method of claim 10, wherein displaying both the ultrasound image and the photographic image on the display device comprises displaying the ultrasound clip in time synchronization with the video clip on the display device.

12. The method of claim 1, further comprising editing the photographic image after associating the photographic image with the ultrasound image, wherein editing comprises performing at least one of adjusting a zoom level, cropping, adjusting a level of contrast, and rotating.

13. The method of claim 1, wherein the photographic image comprises an image of an optical code associated with at least one of a patient or a medical device.

14. The method of claim 1, wherein acquiring the photographic image comprises acquiring the photographic image with a photographic imaging system that is an integral component of the ultrasound imaging system.

15. The method of claim 1, wherein acquiring the photographic image comprises acquiring the photographic image with a photographic imaging system that is separate from the ultrasound imaging system.

16. An ultrasound imaging system comprising:
a probe;
a display device; and
a processor in electronic communication with the probe and the display device, wherein the processor is configured to:
control the probe to acquire an ultrasound image;
receive a photographic image from a photographic imaging device;
associate the photographic image with the ultrasound image; and
display both the photographic image and the ultrasound image on the display device after associating the photographic image with the ultrasound image.

17. The ultrasound imaging system of claim 16, wherein the photographic imaging device is an integral component of the ultrasound imaging system.

18. The ultrasound imaging system of claim 16, wherein the photographic imaging device is a separate component from the ultrasound imaging system.

19. The ultrasound imaging system of claim 18, wherein the photographic imaging device comprises a smartphone or a tablet, and wherein the photographic imaging device is adapted to transmit the photographic image to the ultrasound imaging system with a wireless protocol.

20. The ultrasound imaging system of claim 16, wherein the processor is configured to store the photographic image as a first DICOM object with a first DICOM header and the ultrasound image as a second DICOM object with a second DICOM header, and wherein DICOM header information in the first DICOM header and second DICOM header is used to associate the photographic image with the ultrasound image.

21. The ultrasound imaging system of claim 16, wherein the photographic image comprises a video clip and wherein the ultrasound image comprises an ultrasound clip, and wherein the processor is configured to associate a first series of time stamps with the video clip and a second series of time stamps with the ultrasound clip.

22. The ultrasound imaging system of claim 21, wherein the processor is configured to display the ultrasound clip in time synchronization with the video clip on the display device based on the first series of time stamps and the second series of time stamps.
